(19) United States
(12) Patent Application Publication
(10) Pub. No.: US 2026/0017793 A1
(43) Pub. Date: Jan. 15, 2026

(54) COMPUTER-AIDED SYSTEM AND METHOD FOR DETERMINATION OF AUTISM SPECTRUM DISORDER SEVERITY BY ASSESSMENT MODULE

(71) Applicant: University of Louisville Research Foundation, Inc., Louisville, KY (US)

(72) Inventors: Ayman S. El-Baz, Louisville, KY (US); Mohamed Khudri, Louisville, KY (US); Yaser Elnakieb, Louisville, KY (US); Ahmed Shalaby, Louisville, KY (US); Ali Mahmoud, Louisville, KY (US); Gregory N. Barnes, Louisville, KY (US); Mostafa Abdelrahim, Louisville, KY (US); Mohamed T. Ali, Louisville, KY (US); Ahmed Elnakib, Louisville, KY (US)

(73) Assignee: University of Louisville Research Foundation, Inc., Louisville, KY (US)

(21) Appl. No.: 19/265,967

(22) Filed: Jul. 10, 2025

Related U.S. Application Data
(60) Provisional application No. 63/670,276, filed on Jul. 12, 2024.

(51) Int. Cl. G06T 7/00; A61B 5/00; A61B 5/055; G01R 33/56; G01R 33/563; G06T 7/11; G06T 7/143; G06V 10/764; G06V 20/70; G06V 40/10

(52) U.S. Cl. CPC G06T 7/0014; A61B 5/0042; A61B 5/055; A61B 5/4076; A61B 5/7246; A61B 5/7264; G01R 33/5608; G01R 33/56341; G06T 7/11; G06T 7/143; G06V 10/764; G06V 20/70; G06T 2207/20128; G06T 2207/30016; G06V 40/10; G06V 2201/031

(57) ABSTRACT

A non-invasive computer-aided system and method for assessing the severity of autism spectrum disorders across multiple assessment modules use as input neuroimaging data of a subject brain, parcellates the subject brain into a plurality of brain regions, identifies neuroimaging markers denoting connectivity between regions, determines connectivity between regions, identifies regions associated with autism spectrum disorder, and uses machine learning techniques to determine the severity of autism spectrum disorder with respect to each module based on the determined connectivity.

### CROSS-REFERENCE TO RELATED APPLICATION

This application claims the benefit of priority to U.S. provisional patent application Ser. No. 63/670,276, filed Jul. 12, 2024, for COMPUTER-AIDED SYSTEM AND METHOD FOR DETERMINATION OF AUTISM SPECTRUM DISORDER SEVERITY BY ASSESSMENT MODULE, incorporated herein by reference.

### FIELD OF THE INVENTION

A non-invasive computer-aided system and method for assessing the severity of autism spectrum disorders across multiple assessment modules use as input neuroimaging data of a subject brain, parcellates the subject brain into a plurality of brain regions, identifies neuroimaging markers denoting connectivity between regions, determines connectivity between regions, identifies regions associated with autism spectrum disorder, and uses machine learning techniques to determine the severity of autism spectrum disorder with respect to each module based on the determined connectivity.

### BACKGROUND OF THE INVENTION

Autism spectrum disorder (ASD) is a neurodevelopmental disorder characterized by three primary characteristics: difficulties with social interaction, communication barriers, and behavioral restrictions and repetitive patterns. According to the Centers for Disease Control and Prevention in the United States (CDC), there has been an obvious increase in the prevalence of ASD in recent years. In 2023, the prevalence had risen to one in 36 children, which is a significant 317% increase over the 2000 data. The primary concerns regarding the current assessment tools for ASD, such as the Autism Diagnostic Observation Schedule (ADOS), are their subjectivity, particularly in grading the severity level of each behavioral module of ASD. Additionally, assessment tools are often used late, which limits the effectiveness of interventions. These limitations have motivated neuroscience research to explore developing non-invasive technologies based on medical imaging or genome markers. Despite the lack of a comprehensive understanding of ASD causes, numerous hypotheses and theories have been proposed concerning the etiology of its underlying mechanism. These hypotheses and theories suggest that genes and environmental factors play a significant role in determining ASD severity. Anatomical abnormalities of the brain, functioning of the brain during rest or while performing different tasks, or abnormal connectivity of the white matter are hypothesized to be responsible for ASD symptoms. Several magnetic resonance imaging (MRI)-based imaging methods have been utilized to study a variety of abnormalities correlated with ASD, including diffusion tensor imaging (DTI) for abnormalities in connectivity. DTI is a technique that detects how water travels along white matter tracts in the brain. Unlike MRI-based imaging methods such as fMRI, DTI provides information on static anatomy and is not influenced by brain functions (i.e., performing or not performing tasks).

Current research work in the field of ASD classification based on DTI imaging has significant limitations, generating binary diagnoses distinguishing only between ASD and typically developing brains. However, clinicians require more than just binary classification; they necessitate severity grading to accurately position autistic individuals within the ASD spectrum. This nuanced classification is crucial for guiding interventions and treatments effectively. In addition, clinicians require a more comprehensive evaluation of the five primary ASD assessment modules (communication, motivation, cognition, awareness, and mannerisms), which are crucial for selecting the appropriate ASD intervention while minimizing costs, especially in developed countries where health insurance may not be affordable for all patients. Accordingly, a need exists for an efficient computer-aided diagnosis (CAD) system capable of not only distinguishing between ASD and typically developing brains, but capable of classifying ASD in terms of each module.

### SUMMARY

The instant subject matter relates to assessing the severity of autism spectrum disorders across multiple assessment modules via a system or method that uses as input neuroimaging data of a subject brain, parcellates the subject brain into a plurality of brain regions, identifies neuroimaging markers denoting connectivity between regions, determines connectivity between regions, identifies regions associated with autism spectrum disorder, and uses machine learning techniques to determine the severity of autism spectrum disorder with respect to each behavioral module based on the determined connectivity.

In some embodiments, the present invention includes the following steps. First, DTI image data of a subject brain is preprocessed, followed by extracting the white matter (WM) regions from the image data. Next, the extracted WM regions are identified and parcellated by aligning the extracted WM regions from the subject brain with an existing brain region atlas. Next, for each WM parcellated region, specific DTI markers denoting connectivity in the WM are identified, including, in some embodiments, fractional anisotropy (FA), mean diffusivity (MD), radial diffusivity (RD) and axial diffusivity (AD). Next, a machine learning technique, such as recursive feature elimination (RFE), is used to identify WM brain regions associated with ASD. Next, RFE is applied to the identified regions associated with ASD to find regions that are correlated with each assessment module. Logistic regression is then used to classify the subject brain as ASD or typical development (TD), determining both the overall ASD severity and the ASD severity with respect to each module. In certain embodiments, instead of a single-phase machine learning approach for classifying the subject brain and determining overall ASD severity and ASD severity by assessment module, the system includes a two-phase approach first performing ASD diagnosis and severity determination by module and second integrating these module-specific findings to generate an overall diagnosis and overall determination of ASD severity. The CAD system provides an accurate state-of-the-art diagnosis of ASD and classification of ASD per assessment module based on non-invasive DTI image data of a subject brain. While a human physician may make the ultimate diagnosis, the CAD system can provide an informed prediction of the diagnosis to aid the physician's diagnosis.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following description, in conjunction with the accompanying drawings, provides a better understanding of the present invention.

FIG. 1 depicts a schematic illustration of the steps of a first embodiment of the present invention, including receiving DTI image data, preprocessing, estimation of WM correlation matrix, extraction of WM regions correlated with ASD, and machine learning (ML) classification.

FIG. 2 is a DTI image of a subject brain (a) and depiction of the DTI image after the preprocessing steps of (b), noise reduction, (c) eddy current and motion correction, (d) brain extraction, (e) WM extraction, and (f) WM parcellation.

FIG. 3 is a visual diagram illustrating Pearson correlation values for WM regions.

FIG. 4 is a schematic illustration of the WM correlation matrix estimation procedure.

FIG. 5 illustrates the ASD-correlated regions of a subject brain. Panel (a) is a circular graph with lines describing the connectivity between brain regions. Panel (b) is a correlation matrix with colored cells describing the connectivity between brain regions. Panel (c) is a schematic illustration of the subject brain with regions identified in panels (a) and (b) indicated by color.

FIG. 6 illustrates the identified brain regions correlated with ASD for each assessment module. Left to right, the modules are awareness, cognition, communication, mannerism, and motivation.

FIG. 7 is a three-part graph illustrating the severity of autism within each assessment module for an individual with severe ASD using the system of the first embodiment of the disclosed CAD system. The Y-axis of the graph is the predictive score generated by the CAD system. The graph is divided into three sections, left-to-right, into probabilities that the subject brain is mild ASD vs TD, that the subject brain is moderate ASD vs TD, and that the subject brain is severe ASD vs TD. The score is shown with respect to each assessment module (awareness, cognition, communication, mannerism, motivation), together with a “total report” indicating the probability that the subject brain should be categorized in that section (mild, moderate, or severe ASD).

FIG. 8 is a three-part graph as in FIG. 7, but illustrating a TD individual.

FIG. 9 depicts a schematic illustration of the steps of a second embodiment of the present invention, including receiving DTI image data, preprocessing, estimation of WM correlation matrix, extraction of WM regions correlated with ASD, and two-phase machine learning (ML) classification.

FIG. 10 is a three-part graph illustrating the severity of autism within each assessment module for an individual with severe ASD using the system of the second embodiment of the disclosed CAD system. The Y-axis of the graph is the predictive score generated by the CAD system. The graph is divided into three sections, left-to-right, into probabilities that the subject brain is mild ASD vs TD, that the subject brain is moderate ASD vs TD, and that the subject brain is severe ASD vs TD. The score is shown with respect to each assessment module (awareness, cognition, communication, mannerism, motivation).

FIG. 11 illustrates the identified brain regions correlated with ASD for each assessment module specific to the individual represented in FIG. 10.

FIG. 12 is a three-part graph as in FIG. 10, but illustrating a TD individual.

FIG. 13 illustrates the identified brain regions correlated with ASD for each assessment module specific to the individual represented in FIG. 12.

### DETAILED DESCRIPTION OF THE PREFERRED EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount is meant to encompass variations of ±10% of the most precise digit in the value or amount (e.g., “about 1” refers to 0.9 to 1.1, “about 1.1” refers to 1.09 to 1.11, etc.).

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The present invention relates to a computer-aided diagnostic system and method for determination of ASD severity by assessment module (assessment modules are alternatively referred to as “domains” or “behavioral domains”). While the present invention is discussed in terms of a computer-aided system for diagnosis of ASD, it should be understood that the methodology described herein may be used with other neurological disorders, such as, for example, attention-deficit/hyperactivity disorder (ADHD), schizophrenia, or others.

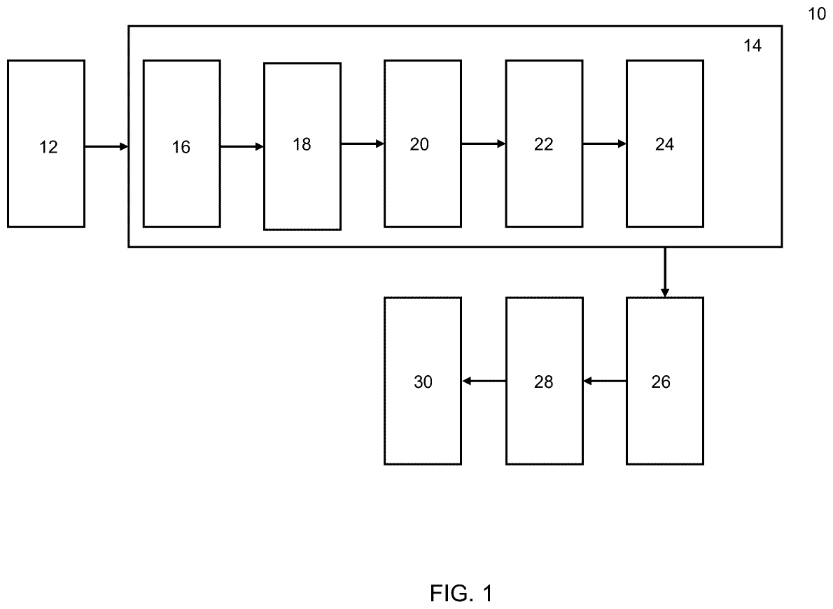

Referring now to FIG. 1, a first embodiment of disclosed CAD system and method (10) is summarized in schematic form. The system and method begin with an initial receiving step (12) of receiving DTI image data of a subject brain. Next, a multi-stage pre-processing step for the DTI image data (14) includes reducing noise (16), correcting for distortion (18), extracting the white matter (WM) regions from the image data (20), identifying and parcellating and the extracted WM regions by aligning the extracted WM regions from the subject brain with an existing brain region atlas (22) and, for each WM parcellated region, identifying and extracting specific DTI markers denoting connectivity in the WM, including fractional anisotropy (FA), mean diffusivity (MD), radial diffusivity (RD) and axial diffusivity (AD) (24). Next, a correlation step (26) includes identifying correlations between DTI markers in different WM brain regions. Next, an extraction step (28) includes using a machine learning technique, such as recursive feature elimination (RFE), to identify which WM brain regions are associated with ASD. Next, a diagnosis step (30) includes applying a machine learning technique, such as RFE, to the identified brain regions associated with ASD to find correlated regions with each assessment module. Logistic regression is then used to classify the subject brain as ASD or TD, determining the overall severity and the severity with respect to each module. Each step will be discussed in further detail.

### Preprocessing

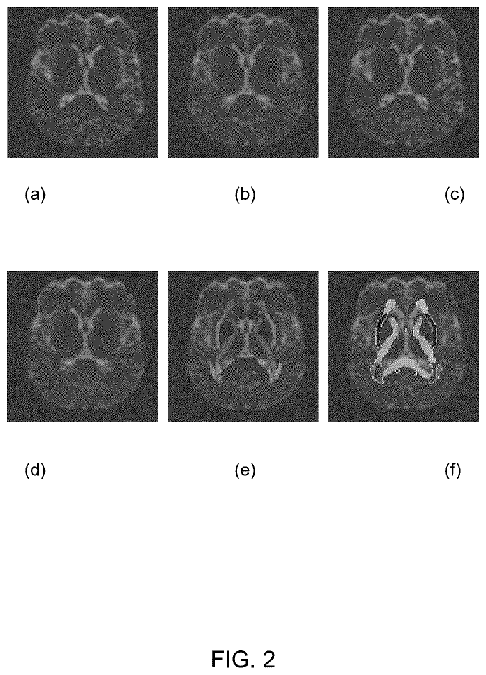

The FSL toolbox, as described in Jenkinson, M., Beckmann, C. F., Behrens, T. E., Woolrich, M. W., Smith, S. M.: Fsl.Neuroimage 62(2), 782-790 (2012), is used to perform standard preprocessing steps on a received DTI image of a subject brain, ensuring the readiness of DTI-collected data for analysis and correlation with ASD markers. An unmodified DTI image of a subject brain is depicted in FIG. 2, panel (a). The first preprocessing step, noise reduction, addresses potential imbalances in MRI magnetic fields. The DTI image subsequent to this step is depicted in FIG. 2, panel (b). Subsequently, the effects of noise stemming from eddy currents and patient movement were mitigated, as depicted in FIG. 2, panel (c). Next, the brain regions were extracted, the WM regions extracted, and then the WM regions registered and parcellated according to the Johns Hopkins atlas as depicted in FIG. 2, panels (d), (e), and (f), respectively. In other embodiments, other brain atlases may be used for registration and parcellation.

After parcellating the WM, DTI radiomics, i.e., quantitative metrics extracted from medical images, are extracted and can be utilized to investigate the microstructural properties of WM by measuring the diffusion of water molecules within it. In one embodiment, the following metrics (Eq. 1-Eq. 5) are employed as estimations of water diffusion inside each WM region.

$$FA ( {λ}_{1} , {λ}_{2} , {λ}_{3} ) = \sqrt{\frac{1}{2} ( \frac{{( {λ}_{1} - {λ}_{2} )}^{2} + {( {λ}_{2} - {λ}_{3} )}^{2} + {( {λ}_{1} - {λ}_{3} )}^{2}}{{λ}_{1}^{2} + {λ}_{2}^{2} + {λ}_{3}^{2}} )} \qquad ( 1 )$$

$$MD = \frac{1}{3} {\sum }_{i = 1}^{3} {λ}_{1} \qquad ( 2 )$$

$$AD = {λ}_{1} \qquad ( 3 )$$

$$RDs = [ {λ}_{2} {λ}_{3} ] \qquad ( 4 )$$

$$TSkew = \frac{1}{3} {\sum }_{i = 1}^{3} {( {λ}_{i} - MD )}^{3} \qquad ( 5 )$$

Where λ₁, λ₂ and λ₃ are eigenvalues of the diffusion tensor.

### WM Radiomics Correlated with ASD

In several studies concerning ASD, evidence has emerged indicating alterations in both short and long-connection neurons within the brain responsible respectively for transmitting signals over short distances within regions of the working memory and integrating information across different brain areas. To accurately characterize these abnormalities in both short and long connections, the present invention utilizes a novel 2D matrix that captures the mutual correlation between all parcellated regions of the WM. The utilization of correlation as a metric offers a normalized measure, thus reducing subject variability, and facilitates the assessment of synchronization in DTI radiomics, which serves as a valuable indicator for identifying abnormalities in both short and long connections in individuals with ASD.

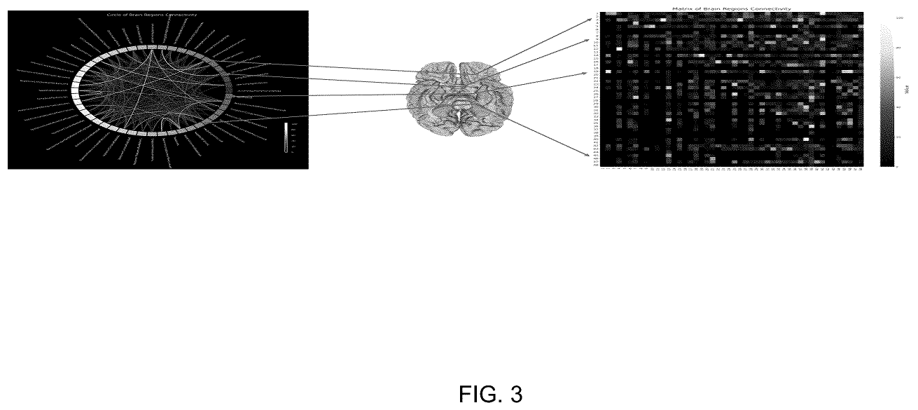

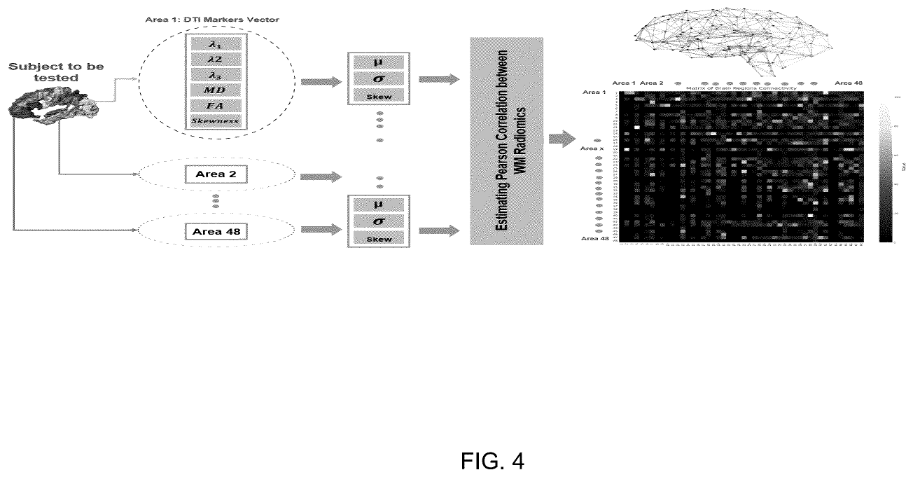

To estimate the 2D mutual correlation matrix, a methodology was employed wherein each DTI radiomic (e.g., FA, MD, RD and AD, as well as TSkew as indicated in Eq. 5), which characterizes water diffusion within specific regions of the parcellated WM, was represented by three metrics. These metrics summarize the statistical properties of water diffusion within each WM region, namely the mean (μ), standard deviation (σ), and skewness (S(x−μ)³/σ³). Consequently, each WM region is characterized by a vector of 15 values, with three values assigned to each DTI radiomic. Then, the Pearson correlation coefficient (Eq. 6) was utilized to compute the Pearson correlation between each pair of regions in the parcellated WM. To facilitate visualization, the Pearson correlation values are represented using specific colors, as illustrated in FIG. 3 and FIG. 4. The estimated number of Pearson correlation coefficients for each subject will be calculated as (48/2×47). Self-comparisons (autocorrelations) are excluded from this calculation because all such values would be 1, offering no discriminatory information between ASD subjects and TD subjects.

$${p}_{i , j} = corr ( s ( i , : ) , s ( j , : ) ) \qquad ( 6 )$$

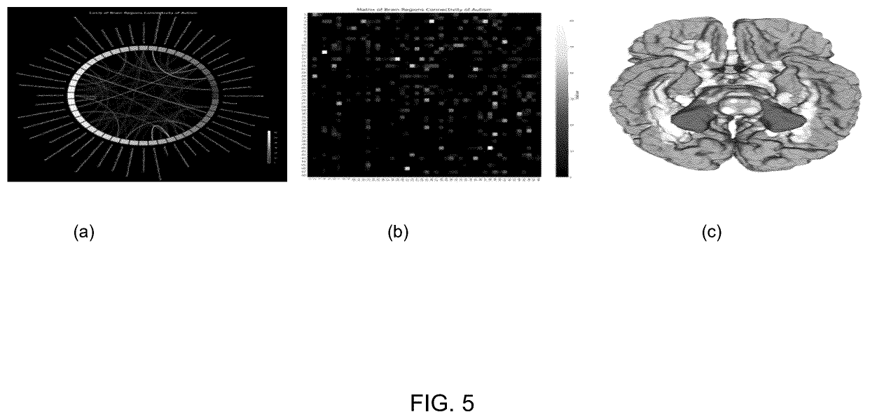

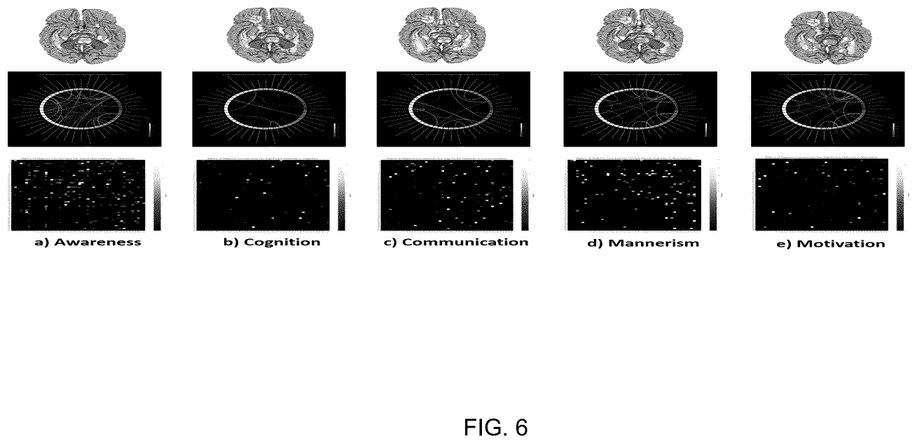

After calculating the correlations between all region pairs, the most significant pairs for each assessment module are selected based on 95% confidence intervals. FIG. 5 illustrates ASD-correlated regions, while FIG. 6 illustrates ASD-correlated regions specifically associated with each assessment module.

### Comprehensive Assessment of ASD

After the regions correlated with ASD and those associated with each ASD module are identified, Logistic Regression (LR) is employed as a classifier, with its parameters hyper-tuned via Bayesian optimization. This method combines iterative decision-making with probabilistic models to streamline optimization (see Algorithm 1). Here, the Social Responsiveness Scale (SRS) assessment tool was utilized as the ground truth. Subjects were classified into four groups based on their scores: (1) Typically Developing, where TD is indicated by score≤59; (2) Mild, where 60≤score<65; (3) Moderate, where 65≤score<76; and (4) Severe, where score≥76. The initial step of the proposed system involves diagnosing individuals into one of the aforementioned classes based on the areas identified to be associated with ASD. The same concept is employed to train a LR classifier for each ASD module, for a total of five LR classifiers, utilizing the identified areas specific to each module, according to Algorithm 1.

Algorithm 1: Machine Learning Training with Optimized Hyperparameter Tuning

Input: WM correlation data matrix and out vector
Output: Trained classifier Model (c)
Steps:

| | |
|---|---|
| 1: | Let X be the WM correlation data matrix comprises c rows, N_{f} are the |

selected columns from RFECV, and y be the output vector of length M such that
y ∈ {0,1}^{M}.

| | |
|---|---|
| 2: | Divide X,y into k folds, i.e., X_{f} = {X₁,X₂,...,Xₖ} and y_{f} = {y₁,y₂,...,yₖ} where |

Xᵢ ∈ R└M/k┘×N_{f} and yi ∈ {0,1}└M/k┘ .

| | |
|---|---|
| 3: | Let c be the set of the C classifiers utilized in the study such that C = |

{c₁,c₂,...,c_{C}} ; In this case it will be LR; For each ci ∈ C, let Hᵢ be the tuple of its
associated hyperparameters HP; For each Hᵢ let v_{i,j} denote the range of allowable
values of element j;

| | |
|---|---|
| 4: | for cᵢ ∈ C do |
| 5: | for HP_setᵢ ∈ {Hᵢ × vᵢ} do |
| 6: | for (Xi,yi) ∈ (Xf,yf) do |
| 7: | Set the hyperparameters of classifier cᵢ to the values |

in HP_setᵢ;

| | |
|---|---|
| 8: | Train classifier cᵢ using (Xₜᵣₐᵢₙ,yₜᵣₐᵢₙ) |
| 9: | Calculate the balanced accuracy score of cᵢ using |

Xₜₑₛₜ,yₜₑₛₜ;

| | |
|---|---|
| 10: | Save the trained classifier cᵢ and its corresponding |

balanced accuracy score if balanced accuracy score >= 80%;

| | |
|---|---|
| 11: | end for |
| 12: | Save the calculated average balanced accuracy score of all |

the iterations along with the corresponding HP_setᵢ;

| | |
|---|---|
| 13: | end for |
| 14: | Save the maximum of all average balanced accuracy scores and |

the corresponding HP_setᵢ, along with classifier cᵢ;

| | |
|---|---|
| 15: | end for |
| 16: | for ci ∈ C do |
| 17: | return the maximum score and the corresponding HP_setᵢ; |
| 18: | end for |

### Results

The system and method disclosed herein were evaluated using 126 subjects with ASD and 100 TD subjects from five neurological institutes identified in the publicly available ABIDE II dataset. The disclosed system and method include two steps. Initially, it assesses severity levels (mild, moderate, severe) across the five modules (communication, motivation, cognition, awareness, and mannerisms). Table 1 shows the determined system accuracy for each module. A comprehensive diagnostic report is then used to establish if the case is classified as TD or ASD.

TABLE 1
Performance of Logistic Regression
(LR) Through Training and Testing

| | Training | | Testing | |
|---|---|---|---|---|
| | #WM | | #WM | |
| Module | Markers | Accuracy | Markers | Accuracy |
| Awareness | 306 | 0.98 ± 0.01 | 227 | 0.94 ± 0.02 |
| Communication | 186 | 0.97 ± 0.01 | 42 | 0.92 ± 0.03 |
| Mannerism | 337 | 0.97 ± 0.02 | 91 | 0.93 ± 0.04 |
| Cognition | 137 | 0.96 ± 0.03 | 99 | 0.92 ± 0.03 |
| Motivation | 292 | 0.96 ± 0.01 | 57 | 0.91 ± 0.03 |

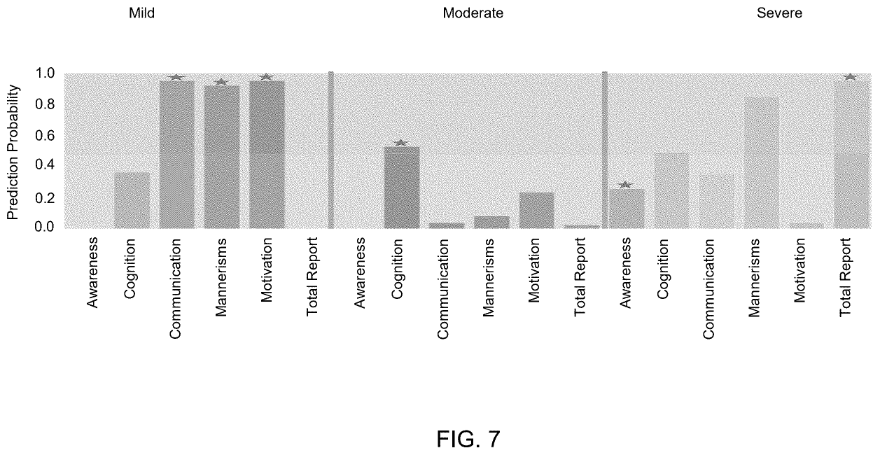

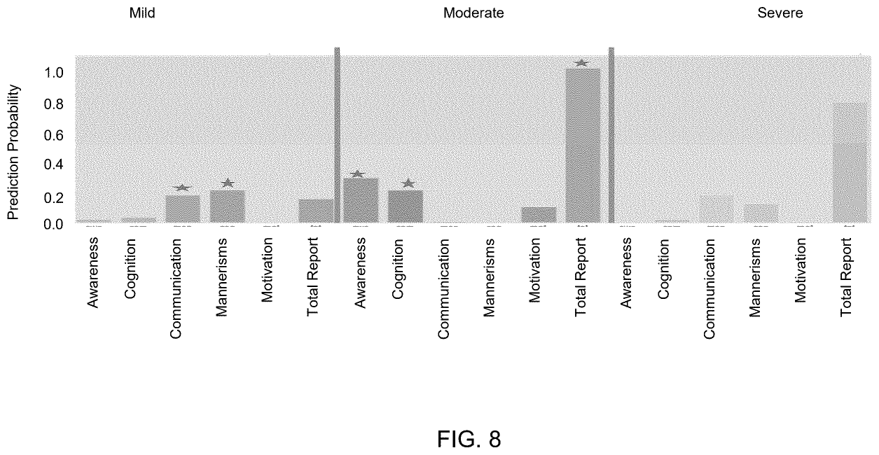

The disclosed system and method perform well with 98% accuracy, 96% sensitivity, and 99% specificity. To highlight the results, graphical representations of two cases studies are presented, one of which is severe ASD (FIG. 7) and the other is typical development (FIG. 8). In both instances, DTI image data of a subject brain is input into the LR classifier trained for each ASD assessment module to be classified as TD, mild ASD, moderate ASD or severe ASD with respect to that module, then a final diagnosis of the subject as ASD or TD is generated based on the classification results from each module. The disclosed system and method generated correct diagnoses for each module in agreement with the behavioral assessment diagnosis SRS for each subject. More specifically, for the subject with severe ASD shown in FIG. 7, the disclosed system generated a prediction of 0.13 TD and 0.87 ASD, indicating the subject should be classified as ASD based on DTI image data of the subject's brain. Bars indicated by stars reflect the clinically determined ASD severity for each assessment module (mild ASD for communication, mannerism and motivation; moderate ASD for cognition; severe ASD for awareness; with a total report of severe ASD). For the TD subject, the disclosed system generated a prediction of 0.22 ASD and 0.78 TD, indicating the subject should be classified as TD based on the DTI image data of the subject's brain. Note that the “Total Report” columns in FIGS. 7 and 8 do not reflect the final diagnosis in each case but are additional behavioral measures derived from the other five assessment modules. Thus, the “Total Report” for each ASD severity level is an engineered feature that represents a combination of the individual modules, rather than a direct indicator of the final diagnosis.

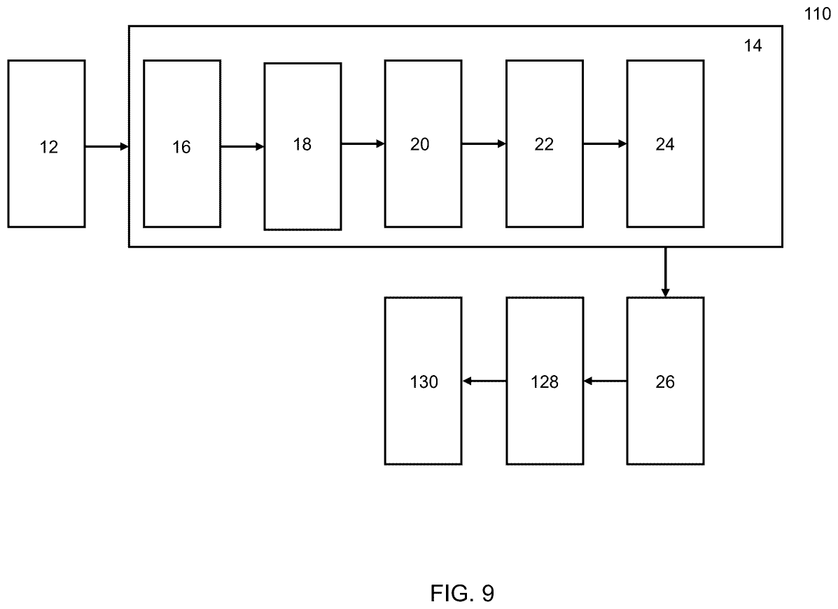

Referring now to FIG. 9, a second embodiment of disclosed CAD system and method (110) is summarized in schematic form. The system and method begins with an initial receiving step (12) of receiving DTI image data of a subject brain and a multi-stage pre-processing step for the DTI image data (14), which includes reducing noise (16), correcting for distortion (18), extracting the white matter (WM) regions from the image data (20), identifying and parcellating and the extracted WM regions by aligning the extracted WM regions from the subject brain with an existing brain region atlas (22) and, for each WM parcellated region, identifying and extracting specific DTI markers denoting connectivity in the WM, including fractional anisotropy (FA), mean diffusivity (MD), radial diffusivity (RD) and axial diffusivity (AD) (24). A correlation step (26) includes identifying correlations between DTI markers in different WM brain regions. While the initial receiving step (12), preprocessing steps (14), and correlation step (26) are the same in the second embodiment 110 as in the first embodiment of the CAD system 10, the remaining steps differ. An extraction step (128) includes a two-phase pipeline including preliminary univariate feature selection followed by RFE to identify which WM brain regions are associated with ASD. Next, a diagnosis step (130) includes applying two-phase machine learning classification system, separately performing ASD severity determination by ASD assessment module followed by a final diagnostic classification. The new extraction and diagnosis steps 128, 130 will be discussed in further detail.

The two-phase extraction step 128 begins with preliminary univariate feature selection to reduce the dimensionality of the feature space before applying model-based selection, thereby improving computational efficiency and reducing overfitting risk. The subject is represented by a feature vector consisting of structural connectivity measures between white matter regions, derived from DTI data. These features capture inter-regional correlation strengths across region pairs. A plurality of statistical methods, including, in some embodiments, (i) analysis of variance (ANOVA F-test), (ii) Chi-square (χ²) test, and (iii) Mutual Information (MI) estimation, are used to identify features with the highest predictive relevance. These tests quantify the statistical dependency between each feature and the target variable (either ASD diagnosis or behavioral severity classification) for the purpose of identifying features that show significant group-level separation or information gain relative to the classification labels. Each feature is scored based on its test statistic (F-value, χ² score, or MI), then ranked. A fixed number or percentile (e.g., top 10%, top 25%, top 50%, etc.) of the highest-ranked features are retained. This filtered subset becomes the input to the next stage of feature selection.

The two-phase extraction step 128 continues using the filtered subset as input into a wrapped-based machine learning method, such as RFE, using a base estimator, such as a linear Support Vector Machine or Logistic Regression model. RFE iteratively trains the model, removes the least important features based on model coefficients, and repeats the process until the optimal subset is found. RFE is preferably coupled with k-fold cross-validation to ensure the stability and generalizability of the selected features. The features selected from this extraction step 128 are then used to train the final machine learning models in both module-specific severity classification and diagnostic classification. Benefits of the two-phase approach include reduced noise and irrelevant information, improved computational efficiency for RFE and downstream classifiers, enhanced generalization performance on limited datasets and improved diagnostic accuracy, as demonstrated in the DTI-system balanced classification accuracy improved to 94%. For clarification, the accuracy data provided in Table 1 in connection with the first embodiment of the invention refers to the accuracy calculated for each assessment module independently from each other, while the accuracy data provided here in connection with the second embodiment refers to the accuracy of the global diagnosis (i.e., ASD or TD) encompassing the holistic assessment of all five modules.

The diagnosis step 130 includes a two-phase machine learning framework that separately performs ASD severity determination by ASD assessment module followed by a final diagnostic classification. First, the system independently evaluates the five assessment modules: social awareness, social cognition, social communication, social motivation, and autistic mannerisms. Each module is treated as a separate multi-class classification task to distinguish between mild, moderate, and severe ASD, each compared against typical development. A separate model is trained for each module using one or more of the following classifiers: linear support vector machine, logistic regression, gradient boosting, and k-nearest neighbor. Each classifier is optimized using cross-validation, and the best-performing model is selected based on balanced classification performance and domain relevance. The output of this first phase is a set of 15 probability scores, three per module, reflecting the likelihood that the subject is mild ASD versus TD, moderate ASD versus TD, or severe ASD versus TD. These probability estimates are used as intermediate diagnostic indicators and passed to the second phase as input.

The second phase of diagnostic step 130 integrates the 15 probability scores to generate a single binary classification of either ASD or TD using a Linear Support Vector Classifier (SVC). After extensive validation testing, SVC achieved the highest balanced classification accuracy as compared to other tested classifiers and ensemble methods. The system outputs a binary ASD diagnosis with an associated confidence score. Together with the domain-level probability breakdowns, these form a comprehensive diagnostic report that supports both clinical decision-making and behavioral profiling.

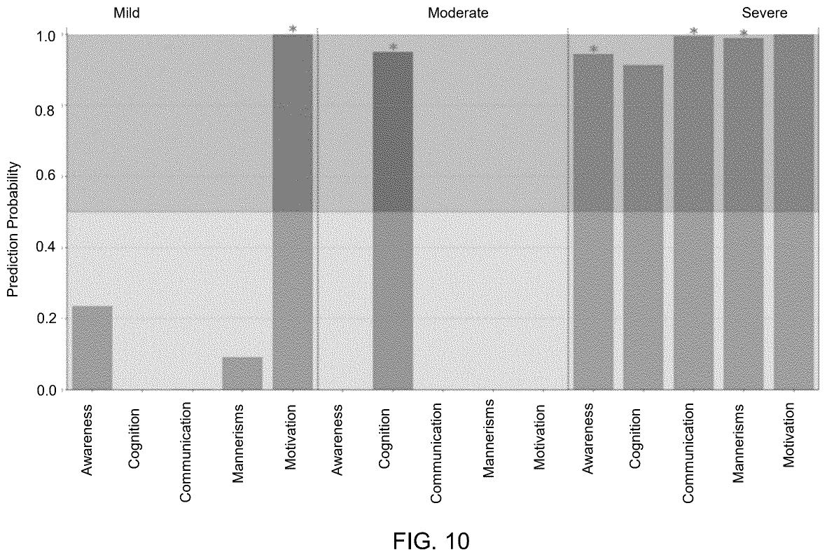

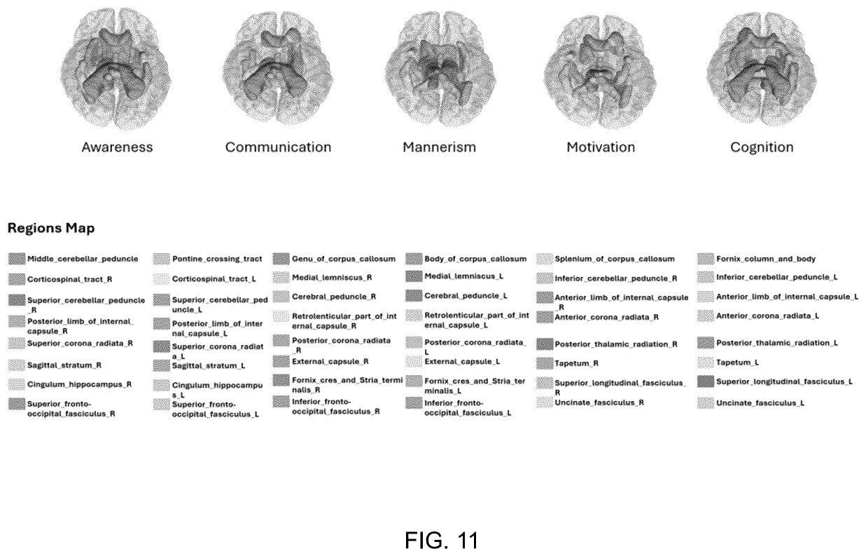

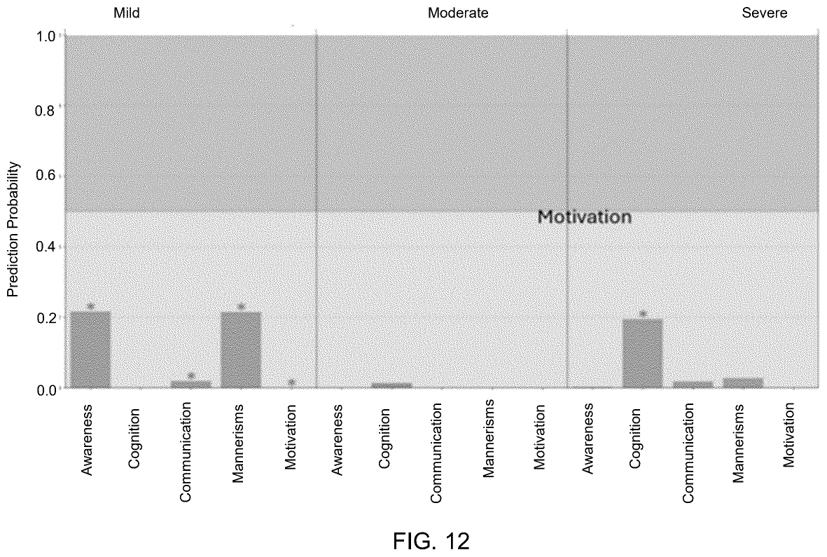

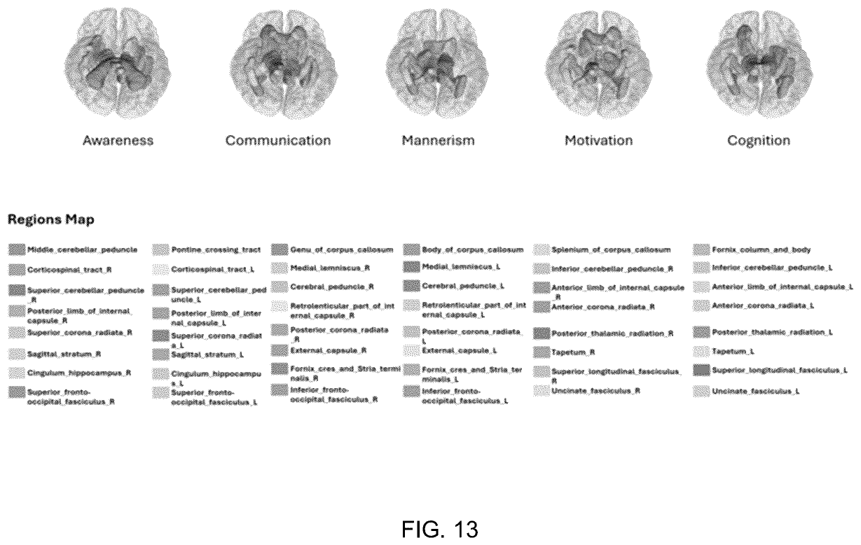

Graphical representations of the results of two additional case studies are shown, one of which is severe ASD (FIGS. 10 and 11) and the other is typical development (FIGS. 12 and 13). In both instances, DTI image data of a subject brain is input into the two-phase classifier trained for each ASD assessment module to be classified as TD, mild ASD, moderate ASD or severe ASD with respect to that module, then, in the second phase, a final diagnosis of the subject as ASD or TD is generated based on the classification results from each module. The disclosed system and method generated correct diagnoses for each module in agreement with the behavioral assessment diagnosis SRS for each subject. More specifically, for the subject with severe ASD shown in FIG. 10, the disclosed system generated a prediction of 1.00 ASD and 0.00 TD, indicating the subject should be classified as ASD based on DTI image data of the subject's brain. Bars indicated by stars reflect the clinically determined ASD severity for each assessment module (mild ASD for motivation (prediction 1.00 mild ASD and 0.00 TD); moderate ASD for cognition (prediction 0.95 moderate ASD, 0.05 TD); severe ASD for awareness (prediction 0.94 severe ASD, 0.06 TD), communication (prediction 0.99 severe ASD, 0.01 TD), and mannerism (prediction 0.99 severe ASD, 0.01 TD)). As a comparison, the ground truth SRS scores for this individual were: awareness 86, cognition 83, communication 86, motivation 76, mannerism 87, and the individual was clinically diagnosed as autistic. FIG. 11 depicts a map of brain regions identified as associated with each assessment module with statistical significance for this individual.

For the TD subject shown in FIG. 12, the disclosed system generated a prediction of 0.00 ASD and 1.00 TD, indicating the subject should be classified as TD based on the DTI image data of the subject's brain. Bars indicated by stars reflect the clinically determined ASD severity for each assessment module (TD for motivation (prediction 0.00 mild ASD and 1.00 TD), cognition (prediction 0.19 severe ASD, 0.81 TD); TD for awareness (prediction 0.22 mild ASD, 0.78 TD), communication (prediction 0.02 mild ASD, 0.98 TD), and mannerism (prediction 0.21 mild ASD, 0.79 TD)). As a comparison, the ground truth SRS scores for this individual were: awareness 52,cognition 41, communication 38, motivation 44, mannerism 47, and the individual was clinically diagnosed as TD. FIG. 13 depicts a map of brain regions identified as associated with each assessment module with statistical significance for this individual.

The disclosed CAD system and method may be embodied in computer program instructions stored on a non-transitory computer-readable storage medium configured to be executed by a computing system. The computing system utilized in conjunction with the CAD system described herein will typically include a processor in communication with a memory, and a network interface. Power, ground, clock, and other signals and circuitry are not discussed, but will be generally understood and easily implemented by those ordinarily skilled in the art. The processor, in some embodiments, is at least one microcontroller or general purpose microprocessor that reads its program from memory. The memory, in some embodiments, includes one or more types such as solid-state memory, magnetic memory, optical memory, or other computer-readable, non-transient storage media. In certain embodiments, the memory includes instructions that, when executed by the processor, cause the computing system to perform a certain action.

The computing system also preferably includes a network interface connecting the computing system to a data network for electronic communication of data between the computing system and other devices attached to the network. In certain embodiments, the processor includes one or more processors and the memory includes one or more memories. In some embodiments, the computing system is defined by one or more physical computing devices as described above. In other embodiments, the computing system may be defined by a virtual system hosted on one or more physical computing devices as described above.

Various aspects of different embodiments of the present disclosure are expressed in paragraphs X1 and X2 as follows:
X1: One embodiment of the present disclosure includes a computer-aided system for diagnosing autism spectrum disorder (ASD), the system comprising: at least one non-transitory computer-readable storage medium having computer program instructions stored thereon; and at least one processor configured to execute the computer program instructions causing the processor to perform the following operations: receiving neuroimaging data of a subject brain; parcellating the neuroimaging data of the subject brain into a plurality of brain regions according to a brain atlas; extracting a plurality of quantitative metrics of the subject brain from the neuroimaging data of each brain region; identifying correlations between the extracted plurality of quantitative metrics from different brain regions; determining which of the plurality of brain regions are associated with ASD based at least in part on the identified correlations; determining which of the brain regions associated with ASD are associated with each of a plurality of assessment modules; and classifying, for each of the plurality of assessment modules, a severity of ASD for that assessment module based at least in part on the identified correlations in brain regions associated with ASD and associated with that assessment module.
X2: Another embodiment of the present disclosure includes a computer-implemented method for diagnosing autism spectrum disorder (ASD), the method comprising: receiving neuroimaging data of a subject brain; parcellating the neuroimaging data of the subject brain into a plurality of brain regions according to a brain atlas; extracting a plurality of quantitative metrics of the subject brain from the neuroimaging data of each brain region; identifying correlations between the extracted plurality of quantitative metrics from different brain regions; determining which of the plurality of brain regions are associated with ASD based at least in part on the identified correlations; determining which of the brain regions associated with ASD are associated with each of a plurality of assessment modules; and classifying, for each of the plurality of assessment modules a severity of ASD for that assessment module based at least in part on the identified correlations in brain regions associated with ASD and associated with that assessment module.

Yet other embodiments include the features described in any of the previous paragraphs X1 or X2 as combined with one or more of the following aspects:

Wherein the neuroimaging data of the subject brain is diffusion tensor imaging data of the subject brain.

Wherein the quantitative metrics comprise at least one of fractional anisotropy, mean diffusivity, radial diffusivity, axial diffusivity, and TSkew.

Wherein the quantitative metrics comprise at least two of, at least three of, or at least four of fractional anisotropy, mean diffusivity, radial diffusivity, axial diffusivity, and TSkew.

Wherein the quantitative metrics comprise fractional anisotropy, mean diffusivity, radial diffusivity, axial diffusivity, and TSkew.

Wherein the step of determining which of the plurality of brain regions are associated with ASD based at least in part on the identified correlations includes an initial step of univariate feature selection to omit less relevant identified correlations followed by a subsequent step of using a machine learning technique to identify a subset of identified correlations as characteristic of ASD from the identified correlations not omitted in the initial step.

Wherein the machine learning technique is recursive feature elimination.

Wherein the step of classifying, for each of the plurality of assessment modules, a severity of ASD for that assessment module is performed using a plurality of machine learning classifiers, each trained to distinguish between different severities of ASD and typical development specific to a different assessment module.

Wherein each of the plurality of machine learning classifiers are trained on the subset of identified correlations.

Wherein each of the plurality of machine learning classifiers are one of a logistic regression classifier, a linear support vector machine, a gradient boosting classifier, and a k-nearest neighbor classifier.

Wherein the classifying the severity of ASD for each of the plurality of assessment modules includes classifying the severity of ASD as one of typical development, mild ASD, moderate ASD or severe ASD for each of the plurality of assessment modules.

Wherein the at least one processor is configured to execute the computer program instructions causing the processor to perform the following additional operation: generating a final diagnosis based at least in part on the classification of the severity of ASD for each of the plurality of assessment modules, wherein the final diagnosis is either autism spectrum disorder or typical development.

Wherein the at least one processor is configured to execute the computer program instructions causing the processor to perform the following additional operation: generating, using a machine learning classifier, a final diagnosis based at least in part on the classification of the severity of ASD for each of the plurality of assessment modules, wherein the final diagnosis is either autism spectrum disorder or typical development, and wherein the machine learning classifier is trained on the subset of identified correlations.

Wherein the machine learning classifier is a support vector machine.

Wherein identifying correlations between the extracted plurality of quantitative metrics from different brain regions includes identifying correlations in statistical properties of water diffusion within each brain region.

Wherein the system or method further comprises generating a graphical visualization of the classification for each assessment module.

Wherein the system or method further comprises generating a final diagnosis based at least in part on the classification of the severity of ASD for each of the plurality of assessment modules, wherein the final diagnosis is either autism spectrum disorder or typical development.

Wherein the method further comprises generating a graphical visualization of the classification for each assessment module.

Wherein the method further comprises generating a final diagnosis based at least in part on the classification of the severity of ASD for each of the plurality of assessment modules, wherein the final diagnosis is either autism spectrum disorder or typical development.

The foregoing detailed description is given primarily for clearness of understanding and no unnecessary limitations are to be understood therefrom for modifications can be made by those skilled in the art upon reading this disclosure and may be made without departing from the spirit of the invention.

1. A computer-aided system for diagnosing autism spectrum disorder (ASD), the system comprising:
at least one non-transitory computer-readable storage medium having computer program instructions stored thereon; and
at least one processor configured to execute the computer program instructions causing the processor to perform the following operations:
receiving neuroimaging data of a subject brain;
parcellating the neuroimaging data of the subject brain into a plurality of brain regions according to a brain atlas;
extracting a plurality of quantitative metrics of the subject brain from the neuroimaging data of each brain region;
identifying correlations between the extracted plurality of quantitative metrics from different brain regions;
determining which of the plurality of brain regions are associated with ASD based at least in part on the identified correlations;
determining which of the brain regions associated with ASD are associated with each of a plurality of assessment modules; and
classifying, for each of the plurality of assessment modules, a severity of ASD for that assessment module based at least in part on the identified correlations in brain regions associated with ASD and associated with that assessment module.

2. The system of claim 1, wherein the neuroimaging data of the subject brain is diffusion tensor imaging data of the subject brain.

3. The system of claim 1, wherein the quantitative metrics comprise at least one of fractional anisotropy, mean diffusivity, radial diffusivity, axial diffusivity, and TSkew.

4. The system of claim 1, wherein the step of determining which of the plurality of brain regions are associated with ASD based at least in part on the identified correlations includes an initial step of univariate feature selection to omit less relevant identified correlations followed by a subsequent step of using a machine learning technique to identify a subset of identified correlations as characteristic of ASD from the identified correlations not omitted in the initial step.

5. The system of claim 4, wherein the machine learning technique is recursive feature elimination.

6. The system of claim 4, wherein the step of classifying, for each of the plurality of assessment modules, a severity of ASD for that assessment module is performed using a plurality of machine learning classifiers, each trained to distinguish between different severities of ASD and typical development specific to a different assessment module.

7. The system of claim 6, wherein each of the plurality of machine learning classifiers are trained on the subset of identified correlations.

8. The system of claim 6, wherein each of the plurality of machine learning classifiers are one of a logistic regression classifier, a linear support vector machine, a gradient boosting classifier, and a k-nearest neighbor classifier.

9. The system of claim 1, wherein the classifying the severity of ASD for each of the plurality of assessment modules includes classifying the severity of ASD as one of typical development, mild ASD, moderate ASD or severe ASD for each of the plurality of assessment modules.

10. The system of claim 1, wherein the at least one processor is configured to execute the computer program instructions causing the processor to perform the following additional operation:
generating a final diagnosis based at least in part on the classification of the severity of ASD for each of the plurality of assessment modules, wherein the final diagnosis is either autism spectrum disorder or typical development.

11. The system of claim 4, wherein the at least one processor is configured to execute the computer program instructions causing the processor to perform the following additional operation:
generating, using a machine learning classifier, a final diagnosis based at least in part on the classification of the severity of ASD for each of the plurality of assessment modules, wherein the final diagnosis is either autism spectrum disorder or typical development, and wherein the machine learning classifier is trained on the subset of identified correlations.

12. The system of claim 1, wherein identifying correlations between the extracted plurality of quantitative metrics from different brain regions includes identifying correlations in statistical properties of water diffusion within each brain region.

13. A computer-implemented method for diagnosing autism spectrum disorder (ASD), the method comprising:
receiving neuroimaging data of a subject brain;
parcellating the neuroimaging data of the subject brain into a plurality of brain regions according to a brain atlas;
extracting a plurality of quantitative metrics of the subject brain from the neuroimaging data of each brain region;
identifying correlations between the extracted plurality of quantitative metrics from different brain regions;
determining which of the plurality of brain regions are associated with ASD based at least in part on the identified correlations;
determining which of the brain regions associated with ASD are associated with each of a plurality of assessment modules; and
classifying, for each of the plurality of assessment modules a severity of ASD for that assessment module based at least in part on the identified correlations in brain regions associated with ASD and associated with that assessment module.

14. The computer-implemented method of claim 13, further comprising generating a graphical visualization of the classification for each assessment module.

15. The computer-implemented method of claim 13, further comprising generating a final diagnosis based at least in part on the classification of the severity of ASD for each of the plurality of assessment modules, wherein the final diagnosis is either autism spectrum disorder or typical development.

16. The computer-implement method of claim 13, wherein identifying correlations between the extracted plurality of quantitative metrics from different brain regions includes identifying correlations in statistical properties of water diffusion within each brain region.

17. The computer-implemented method of claim 13, wherein the neuroimaging data of the subject brain is diffusion tensor imaging data of the subject brain.

18. The computer-implemented method of claim 13, wherein the quantitative metrics comprise at least one of fractional anisotropy, mean diffusivity, radial diffusivity, axial diffusivity, and TSkew.
